(19) United States
(12) Patent Application Publication
(10) Pub. No.: US 2016/0263121 A1
(43) Pub. Date: Sep. 15, 2016

(54) STABLE POWDER FORMULATION CONTAINING AN ANTICHOLINGERIC AGENT

(71) Applicant: Boehringer Ingelheim International GmbH, Ingelheim am Rhein (DE)

(72) Inventors: Michael TRUNK, Ingelheim am Rhein (DE); Claudius WEILER, Ingelheim Grosswinternheim (DE); Werner PIEROTH, Bingen am Rhein (DE)

(21) Appl. No.: 15/157,695

(22) Filed: May 18, 2016

Related U.S. Application Data
(63) Continuation of application No. 13/706,885, filed on Dec. 6, 2012, now abandoned.
Application No. 13/706,885 is a continuation of application No. 12/515,780, filed on Oct. 2, 2009, now abandoned.

(30) Foreign Application Priority Data
Nov. 22, 2006 (EP) 06124580.9

(51) Int. Cl. A61K 31/5375; A61K 9/48; A61K 9/00; A61K 9/16

(52) U.S. Cl. CPC A61K 31/5375; A61K 9/1623; A61K 9/4858; A61K 9/0075; A61K 9/1617

(57) ABSTRACT

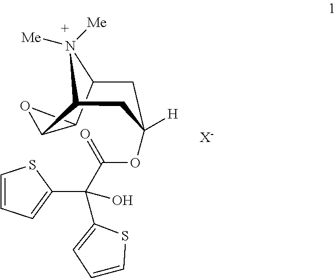

A spray-dried powder formulation comprising particles that contain the following components i) to iii): i) anticholinergic agents, in particular at least one compound of formula 1, in which X⁻ is a negatively charged anion, ii) at least one embedding material selected from the group consisting of mono- or disaccharides, oligosaccharides, polymers, sugar alcohols and cholesterol, and iii) an organic, physiologically acceptable, sterically demanding acid, selected from the group consisting of ascorbic acid, a monovalent, divalent or trivalent carboxylic acid, with the exception of amino carboxylic acids, to preferably fumaric acid, oxalic acid, or diacetic acid, and a fruit acid or culinary acid, preferably citric acid, tartaric acid, malic acid, lactic acid, acetic acid, α-hydroxycaprylic acid or gluconic acid.

### BRIEF SUMMARY OF THE INVENTION

The present invention relates to a spray-dried powder formulation comprising particles which contain the following components i) to iii):
i) anticholinergics, particularly at least one compound of formula 1

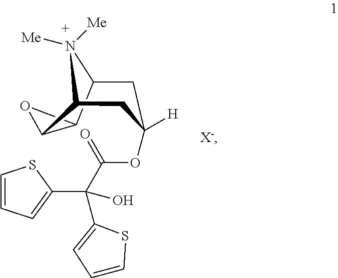

wherein
X⁻ denotes a negatively charged anion.
ii) at least one embedding material selected from among mono- or disaccharides, oligosaccharides, polymers, sugar alcohols and cholesterol,
iii) an organic, physiologically acceptable, sterically demanding acid selected from among ascorbic acid, a mono-, di- or trivalent carboxylic acids with the exception of the aminocarboxylic acids, preferably fumaric acid, oxalic acid or succinic acid, and a fruit or culinary acid, preferably citric acid, tartaric acid, malic acid, lactic acid, acetic acid, α-hydroxycaprylic acid or gluconic acid.

The invention also relates to a process for preparing the above spray-dried powder formulation. In addition, the invention relates to the use of an organic, physiologically acceptable, sterically demanding acid selected from among ascorbic acid, a mono-, di- or trivalent carboxylic acid, with the exception of the aminocarboxylic acids, preferably fumaric acid, oxalic acid or succinic acid, and a fruit or culinary acid, preferably citric acid, tartaric acid, malic acid, lactic acid, acetic acid, α-hydroxycaprylic acid or gluconic acid, preferably citric acid, for stabilising a powder formulation prepared by spray-drying containing
i) anticholinergics, particularly at least one compound of formula 1

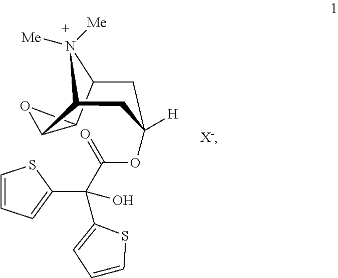

wherein
X⁻ denotes a negatively charged anion.
ii) at least one embedding material selected from among mono- or disaccharides, oligosaccharides, polymers, sugar alcohols and cholesterol.

Compounds of formula 1 are already known from WO 02/30928. They have valuable pharmacological properties and can provide therapeutic benefit as highly effective anticholinergics in the treatment of respiratory complaints, particularly in the treatment of inflammatory and/or obstructive diseases of the respiratory tract, particularly for treating asthma or COPD (chronic obstructive pulmonary disease).

Suitable powder formulations containing at least one compound according to formula 1 have to meet various requirements:
the powder formulations have to contain particles that are “inhalable”, i.e. the particles must have a relatively small mean diameter, preferably ≦10 μm, in order to be “respirable”, i.e. to allow topical application to the lungs
the powder formulation must have sufficient stability when stored at ambient temperature over lengthy periods
homogeneous distribution of the individual components i) to iii) in the particles.

The mean geometric diameter of particles may be determined experimentally for example by a laser diffraction process using a laser made by Sympatec (50 mm focal length) with dry dispersion (Rodos 3 bar).

Consequently, the aim of the present invention was to provide a powder formulation containing an anticholinergic, particularly the compound according to formula 1, as active substance, which meets the conditions outlined above.

### BRIEF DESCRIPTION OF THE DRAWINGS

FIG. 1: An inhaler for the administration of the inhalable powder according to the invention.

### DETAILED DESCRIPTION OF THE INVENTION

In order to prepare a powder formulation there is the option for example of micronizing the active substance, preferably using an air jet mill However, a number of disadvantages are encountered when micronizing the compound according to formula 1. Thus, for example, micronized powder formulations containing a compound according to formula 1 as active substance generally exhibit particle growth after a short time under the effect of moisture, with the result that the particles are only “respirable” under certain conditions. Moreover, when the compound according to formula 1 is micronized, there is the disadvantage that the active substance, i.e. the compound according to formula 1, is not homogeneously distributed in the formulation as a whole. The desired therapeutically effective dose for the compound according to formula 1, at approx. 1% active substance based on the total formulation, is very small and consequently it is hardly possible to achieve uniform distribution of this small amount of active substance in the total formulation simply by micronization and mixing.

Another possible way of preparing a powder formulation is to prepare the formulation by spray-drying. A solution of the active substance and a suitable inert embedding material is preferably atomised using a nozzle and then dried in the hot air current. However, severe problems were also encountered with regard to the chemical stability of the compound according to formula 1 when preparing a powder formulation containing at least one compound of formula 1 as active substance and a suitable embedding material by spray drying. If during the spray drying a solution containing only a compound according to formula 1 and a suitable embedding material is atomised and then dried, this leads to major chemical decomposition of the compound according to formula 1.

Surprisingly, however, it has been found that a spray-dried powder formulation comprising particles which contain the following components i) to iii):
i) an anticholinergic, preferably a compound of formula 1

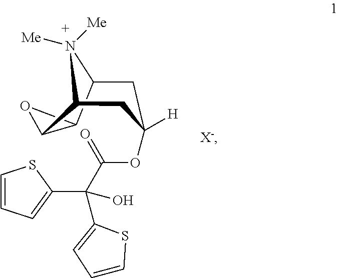

wherein
X⁻ denotes a negatively charged anion.
ii) at least one embedding material selected from among mono- or disaccharides, oligosaccharides, polymers, sugar alcohols and cholesterol,
iii) an organic, physiologically acceptable, sterically demanding acid, preferably selected from among ascorbic acid, a mono-, di- or trivalent carboxylic acid with the exception of the aminocarboxylic acids, preferably fumaric acid, oxalic acid or succinic acid, and a fruit or culinary acid, preferably citric acid, tartaric acid, malic acid, lactic acid, acetic acid, α-hydroxycaprylic acid or gluconic acid are chemically and physically stable for lengthy periods at ambient temperature (and even at temperatures of up to 40° C.).

This improved stability is brought about in particular by the stabilising effect of the organic, physiologically acceptable, sterically demanding acid, preferably selected from ascorbic acid, a mono-, di- or trivalent carboxylic acid with the exception of the aminocarboxylic acids, preferably fumaric acid, oxalic acid or succinic acid, and a fruit or culinary acid, preferably citric acid, tartaric acid, malic acid, lactic acid, acetic acid, α-hydroxycaprylic acid or gluconic acid, on the anticholinergic, preferably on the compound of formula 1. Furthermore, the powder formulation according to the invention has inhalable, i.e. respirable, particle sizes and also a homogeneous distribution of the compound of formula 1 in the particles. Thus, the powder formulation according to the invention meets all the requirements that have to be met by a powder formulation containing a compound of formula 1 as active substance.

The spray-dried powder formulation according to the invention preferably contains, as the organic, physiologically acceptable, sterically demanding acid according to iii), an acid selected from ascorbic acid, a mono-, di- or trivalent carboxylic acid (with the exception of the aminocarboxylic acids), and a fruit or culinary acid.

By a mono-, di- or trivalent carboxylic acid are meant, for the purposes of the invention, C₂- to C₁₀, preferably C₃- to C₆-carboxylic acids with in each case one, two or three carboxyl groups, with the exception of the aminocarboxylic acids, while fumaric acid, oxalic acid, succinic acid, citric acid, tartaric acid, malic acid, lactic acid, acetic acid, α-hydroxycaprylic acid or gluconic acid are preferred and citric acid is particularly preferred.

By culinary or fruit acids are preferably meant, for the purposes of the invention, the acids selected from among citric acid, tartaric acid, malic acid, lactic acid, acetic acid, α-hydroxycaprylic acid or gluconic acid, particularly preferably citric acid.

In another preferred embodiment the spray-dried powder formulation contains the organic, physiologically acceptable, sterically demanding acid according to iii) in the concentration needed to give the sprayable solution containing the anticholinergic according to i), preferably a compound according to formula 1, and the at least one embedding material according to ii), a pH of <7, preferably ≦6, more preferably ≦5, particularly ≦4.

Particularly preferably the spray-dried powder formulation contains citric acid as the organic, physiologically acceptable, sterically demanding acid according to iii) in the concentration needed to give the sprayable solution containing the anticholinergic according to i), preferably a compound according to formula 1, and the at least one embedding material according to ii), a pH of <7, preferably ≦6, more preferably ≦5, particularly ≦4.

Additionally, it has surprisingly been found that the presence of the salt of an organic, physiologically acceptable, sterically demanding acid in addition to the organic, physiologically acceptable, sterically demanding acid according to iii) has a decidedly favourable effect on the chemical stability of the spray-dried powder formulation according to the invention. Consequently, in a particularly preferred embodiment, the above-mentioned spray-dried powder formulation according to the invention additionally contains the salt of an organic, physiologically acceptable, sterically demanding acid, preferably selected from among the ascorbate, the salt of a mono-, di- or trivalent carboxylic acid, with the exception of the salts of the aminocarboxylic acids, preferably the fumarate, oxalate or succinate, and the salt of a fruit or culinary acid, preferably the citrate, tartrate, malate, lactate, acetate, α-hydroxycapronate or gluconate. The citrate is particularly preferred. This additional salt of an organic, physiologically acceptable, sterically demanding acid according to iii) is preferably an alkali metal salt, an alkaline earth metal salt or a zinc salt, preferably an alkali metal citrate, an alkaline earth metal citrate or a zinc citrate, particularly preferably sodium citrate, potassium citrate, magnesium citrate, calcium citrate or zinc citrate.

The additional salt of an organic, physiologically acceptable, sterically demanding acid selected from among the ascorbate, the salt of a mono-, di- or trivalent carboxylic acid with the exception of the salts of the aminocarboxylic acids, preferably the fumarate, oxalate or succinate, and the salt of a fruit or culinary acid, preferably the citrate, tartrate, malate, lactate, acetate, α-hydroxycapronate or gluconate is preferably used in a concentration such that the molar ratio of the anticholinergic according to i), preferably the compound according to formula 1, to the cation of the salt is from 1:1 to 1:12, preferably from 1:2 to 1:10 and particularly from 1:3 to 1:8.

The spray-dried powder formulation according to the invention preferably includes a compound of formula 1, wherein X⁻ is an anion selected from among the chloride, bromide, iodide, sulphate, phosphate, methanesulphonate, nitrate, maleate, acetate, citrate, fumarate, tartrate, oxalate, succinate, benzoate and p-toluenesulphonate, particularly bromide.

In a preferred embodiment the spray-dried powder formulation comprises the anticholinergic, preferably the above-defined compound of formula 1 according to i) in a concentration between 0.01 to 5%, based on the solid, more preferably in a concentration between 0.02 to 2% based on the solid, particularly in a concentration between 0.05 to 1% based on the solid.

In another preferred embodiment the spray-dried powder formulation comprises as embedding material ii) a mono- or disaccharide selected from among glucose, fructose, arabinose, mannitol, saccharose, maltose, lactose, cellobiose and trehalose.

In another preferred embodiment the spray-dried powder formulation comprises as embedding material ii) an oligosaccharide selected from among oligomaltose, oligofructose, cyclodextrins, dextranes, dextrins (e.g. cyclodextrins such as for example α-cyclodextrin, β-cyclodextrin, γ-cyclodextrin, methyl-β-cyclodextrin, hydroxypropyl-β-cyclodextrin) and oligosaccharose.

In another preferred embodiment the spray-dried powder formulation comprises as embedding material ii) a polymer selected from among inulin, alginate, maltodextrin, starch, starch derivatives, cellulose, cellulose derivatives, PVP (plasdone), gelatine, chitosan, dextranes, pectins, gum arabic, polylactides, poly(lactide-co-glycolides) and polyvinylalcohols.

In another preferred embodiment the spray-dried powder formulation comprises as embedding material ii) a sugar alcohol selected from among mannitol, xylitol and sorbitolol.

In another preferred embodiment the spray-dried powder formulation comprises cholesterol as embedding material ii).

In another preferred embodiment the spray-dried powder formulation according to the invention comprises in addition to the constituents i) to iii) other physiologically acceptable excipients. Examples of physiologically acceptable excipients which may be used to prepare the spray-dried powder formulations according to the invention, include for example salts, e.g. sodium chloride, potassium chloride etc., particularly, but not exclusively in the form of their hydrates, complexing agents, flavourings, preservatives and vitamins

In another preferred embodiment the spray-dried powder formulation according to the invention comprises particles that have a median aerodynamic diameter of ≦15 μm, preferably ≦10 μm, particularly ≦5 μm. Particularly preferred are spray-dried powder formulations according to the invention which contain particles that are “inhalable”. “Inhalable particles” or “respirable particles” within the scope of the present invention means that these particles have a median aerodynamic diameter which is small enough to achieve topical application to the lungs. This is the case particularly when the particles have a median aerodynamic diameter ≦10 μm.

The median aerodynamic diameter (MMAD) may be determined experimentally by the cascade impactor method which is described in the European Pharmacopeia, in the 2000 Supplement on determining the MMAD.

The compound of formula 1 is preferably administered by inhalation. As well as inhalable solutions it is also possible to use suitable inhalable powders, which are preferably packed into suitable capsules (inhalettes) and administered using corresponding powder inhalers.

The inhalable powders according to the invention may for example be administered using inhalers that deliver a single dose from a reservoir using a measuring chamber (e.g., as in U.S. Pat. No. 4,570,630A) or by other types of apparatus (e.g. As in DE 36 25 685 A).

Alternatively, and equal importantly according to the invention, the inhalable powders according to the invention may also be administered using inhalers which contain the inhalable powder in a number of individually packaged doses (Pre-Metered Dry Powder Inhaler). The number of individually packaged doses may be provided in the form of a Multi-Dose Blister and particularly in the form of a circular disc which may hold a number of single doses of powder in wells arranged in a circle. Alternatively the plurality of individually packaged doses may also be arranged in the form of a blister strip.

Alternatively, and equal importantly according to the invention, the inhalable powders according to the invention may also be packed into capsules which are used in inhalers as described for example in WO 94/28958.

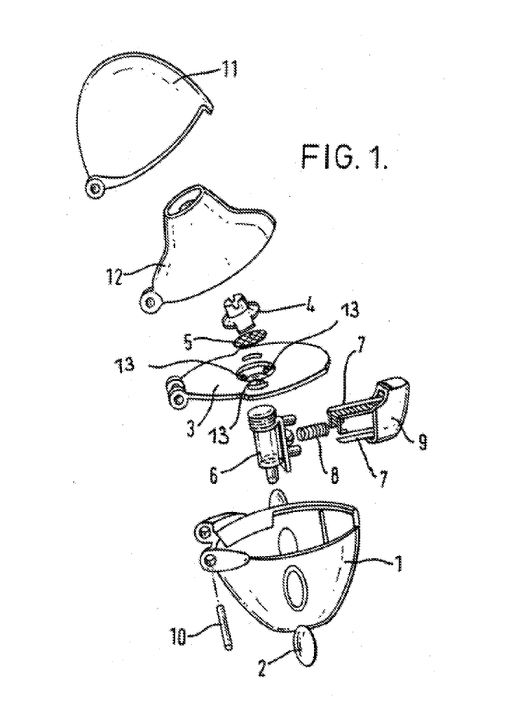

Preferably the capsules containing the inhalable powder according to the invention are administered using an inhaler as shown in FIG. 1.

This inhaler is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut and air holes 13 for adjusting the flow resistance. In another preferred embodiment the powder formulation according to the invention is used in an inhaler according to U.S. Pat. No. 5,590,645. The contents of U.S. Pat. No. 5,590,645 are hereby incorporated by reference. U.S. Pat. No. 5,590,645 describes an inhalation device for using a medicament package in which at least one container for a pharmaceutical powder composition is defined by two removal papers.

In another preferred embodiment the powder formulation according to the invention is used in an inhaler according to U.S. Pat. No. 4,627,432. The contents of U.S. Pat. No. 4,627,432 are hereby incorporated by reference. U.S. Pat. No. 4,627,432 describes an inhaler for administering medicaments to patients, which comprises a housing containing a cylindrical chamber for receiving a carrier, e.g. a blister pack. The blister pack comprises a plurality of containers or blisters which are arranged in a circle. Once the blister pack has been received by the holder, its blisters are located in holes in the holder. A plunger is arranged so as to be able to penetrate through the hole into the chamber, thereby opening a blister contained therein.

When the blister is opened, the patient can remove the medicament through the mouthpiece by inhalation. An external element is used to rotate the holder so that the next blister can come into contact with the piston. Air can enter the chamber through a hole in the cover, which is removable to allow the blister pack to be loaded into the chamber in the holder.

In another preferred embodiment the powder formulation according to the invention is used in an inhaler according to WO 95/16483. The content of WO 95/16483 is hereby incorporated by reference. WO 95/16483 describes an inhaler for delivering doses of a pharmaceutical powder composition, which contains a housing with a cylindrical container. The container has a plurality of helically arranged compartments, all of which hold a dose of the pharmaceutical composition. To release the pharmaceutical composition from a compartment, this compartment has to be placed in the air pathway of the inhaler using an indexing mechanism and the user sucks through the mouthpiece of the housing, this mouthpiece communicating with the air outlet of the air pathway. The flow of air through the air pathway releases the single dose of material. The container may be a replaceable cartridge.

In another preferred embodiment the powder formulation according to the invention is used in an inhaler according to WO 95/31238. The content of WO 95/31238 is hereby incorporated by reference. WO 95/31238 describes an inhaler for delivering single doses of a pharmaceutical powder composition, which has a housing for receiving a container, the container having a plurality of sealed openings containing individually encapsulated doses of a medicament. The container may be movable relative to the housing in order to bring each open successively into the air pathway which communicates with the mouthpiece. The inhaler contains a piercing device, e.g., a bolt, which can be inserted in a selected opening so as to break open the corresponding seal. The configuration and movement of the bolt are coordinated such that almost no powder is expelled in the process.

In another preferred embodiment the powder formulation according to the invention is used in an inhaler according to WO 02/26302. The content of WO 02/26302 is hereby incorporated by reference. WO 02/26302 describes an inhaler for delivering single doses of a pharmaceutical powder composition, which has an air pathway through which the dose migrates from one ejection zone to the outlet of the air pathway. The air pathway has an inlet device which is arranged so as to form an air pocket which flows through part of the air pathway and extends from the ejection zone to the outlet. The air pocket surrounds the said dose and thereby prevents it from hitting the walls of the air pathway. This reduces the accumulation of the material on the walls of the air pathway and in this way the consistency of the performance of the inhaler is improved. Preferably the inlet device has a neck for producing a current of fast-flowing air, thereby forming a low pressure zone in front of the ejection zone, thus making the ejection of the dose easier.

In another preferred embodiment the powder formulation according to the invention is used in an inhaler according to WO 05/002654. The contents of WO 05/002654 are hereby incorporated by reference.

WO 05/002654 describes an inhaler for delivering separate individual doses of a pharmaceutical powder composition from corresponding pouches in a disc-shaped carrier by destroying a cover film from the outside, by applying pressure to the opposite surface. The inhaler has correspondingly individual disaggregation processes for each pouch, split air currents which allow improved flow of the pharmaceutical composition, a switching mechanism for the external destruction of the pouches and a counter for the individual doses.

The invention further relates to a process for preparing a powder formulation that contains particles, comprising the following steps:
a) preparing a solution comprising components i) to iii):
   i) an anticholinergic, preferably a compound of formula 1

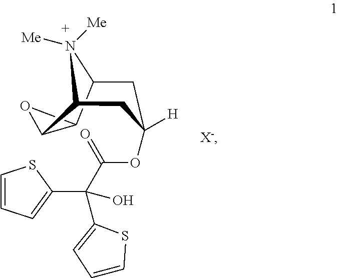

wherein
X⁻ denotes a negatively charged anion.
   ii) at least one embedding material selected from among mono- or disaccharides, oligosaccharides, polymers, sugar alcohols and cholesterol,
   iii) an organic, physiologically acceptable, sterically demanding acid, preferably selected from among ascorbic acid, a mono-, di- or trivalent carboxylic acid with the exception of the aminocarboxylic acids, preferably fumaric acid, oxalic acid or succinic acid, and a fruit or culinary acid, preferably citric acid, tartaric acid, malic acid, lactic acid, acetic acid, α-hydroxycaprylic acid or gluconic acid in a suitable solvent;
b) atomising the solution from step a) in a hot air current
c) drying the atomised solution from step b) to form a powder containing particles
d) separating off the powder from step c) using a cyclone and/or filter

Any current spray-drying apparatus in which steps a) to d) are carried out may be used for the process according to the invention.

In step a) a solution of components i) to iii) in a suitable solvent is prepared.

Preferably X⁻ in the compound of formula 1 denotes an anion selected from among the chloride, bromide, iodide, sulphate, phosphate, methanesulphonate, nitrate, maleate, acetate, citrate, fumarate, tartrate, oxalate, succinate, benzoate and p-toluenesulphonate, particularly bromide.

The solution from step a) preferably contains as the organic, physiologically acceptable, sterically demanding acid according to iii) an acid selected from among ascorbic acid, a mono-, di- or trivalent carboxylic acid, with the exception of the aminocarboxylic acids, and a fruit or culinary acid.

By a mono-, di- or trivalent carboxylic acid are meant, for the purposes of the invention, C₂- to C₁₀, preferably C₃-to C₆-carboxylic acids with in each case one, two or three carboxyl groups with the exception of the aminocarboxylic acids, while fumaric acid, oxalic acid, succinic acid, citric acid, tartaric acid, malic acid, lactic acid, acetic acid, α-hydroxycaprylic acid or gluconic acid are preferred and citric acid is particularly preferred.

By culinary or fruit acids are preferably meant, for the purposes of the invention, the acids selected from among citric acid, tartaric acid, malic acid, lactic acid, acetic acid, α-hydroxycaprylic acid or gluconic acid, particularly preferably citric acid.

In another preferred embodiment of the process according to the invention, a sufficient quantity of the organic, physiologically acceptable, sterically demanding acid according to iii) is added to the solution from step a), which already contains the anticholinergic according to i), preferably a compound according to formula 1, and at least one embedding material according to ii), such that before the atomising according to step b) the solution has a pH of <7, preferably ≦6, more preferably ≦5, particularly ≦4.

In a particularly preferred embodiment of the process according to the invention, a sufficient quantity of citric acid is added to the solution from step a), which already contains the anticholinergic according to i), preferably a compound according to formula 1, and at least one embedding material according to ii), such that before the atomising according to step b) the solution has a pH of <7, preferably ≦6, more preferably ≦5, particularly ≦4.

In another particularly preferred embodiment, the solution from step a) contains in addition to components i) to iii) the salt of an organic, physiologically acceptable, sterically demanding acid according to iii), preferably selected from among the ascorbate, the salt of a mono-, di- or trivalent carboxylic acid, with the exception of the salts of the aminocarboxylic acids, preferably the fumarate, oxalate or succinate, and the salt of a fruit or culinary acid, preferably the citrate, tartrate, malate, lactate, acetate, α-hydroxycapronate or gluconate. The citrate is particularly preferred. This additional salt of an organic, physiologically acceptable, sterically demanding acid according to iii) is preferably an alkali metal salt, an alkaline earth metal salt or a zinc salt, preferably an alkali metal citrate, an alkaline earth metal citrate or a zinc citrate, particularly preferably sodium citrate, potassium citrate, magnesium citrate, calcium citrate or zinc citrate.

Preferably, the ratio of the organic, physiologically acceptable, sterically demanding acid, preferably citric acid, to the salt of the organic, physiologically acceptable, sterically demanding acid, preferably citrate, in the solution is adjusted such that the solution prepared in step a) containing an anticholinergic according to i), preferably a compound of formula 1 and at least one embedding material according to ii), has a pH of <7, preferably ≦6, particularly ≦5, more preferably ≦4.

Preferably in step a) the concentration of the salt of the organic, physiologically acceptable, sterically demanding acid used is such as to obtain a molar ratio of the anticholinergic according to i), particularly the compound of formula 1, to the cation of the salt of 1:1 to 1:12, preferably from 1:2 to 1:10 and particularly from 1:3 to 1:8. Preferably, the embedding material ii) used is a mono- or disaccharide selected from among glucose, saccharose, fructose, maltose, lactose, cellobiose and trehalose, an oligosaccharide selected from among oligomaltose, oligofructose, cyclodextrins, dextrins and oligosaccharose, a polymer selected from among inulin, alginate, maltodextrin, starch, starch derivatives, cellulose, cellulose derivatives, PVP (plasdone), gelatine, chitosan, dextranes, pectins, gum arabic, polylactides, poly(lactide-co-glycolides) and polyvinylalcohols, a sugar alcohol selected from among mannitol, xylitol and sorbitolol or cholesterol. However, the particularly preferred embedding materials ii) used are lactose, trehalose and mannitol, particularly lactose.

Preferably the compound of formula 1 according to i) is dissolved in the solvent in a concentration of 0.04 g/100 ml to 0.4 g/100 ml, the embedding material according to ii) is dissolved in the solvent in a concentration of 5 g/100 ml to 15 g/100 ml and the organic, physiologically acceptable, sterically demanding acid according to iii) is added in a concentration such as to obtain a pH of <7, preferably ≦6, more preferably ≦5, particularly ≦4.

The solvent used may be water, any suitable organic solvent, a mixture of water and organic solvent and a mixture of different organic solvents. It is preferable to use water, ethanol and an ethanol/water mixture as solvent.

In a particularly preferred embodiment of the process according to the invention the atomising of the solution from step a) into step b) is carried out using a nozzle. This nozzle is preferably operated by fluid pressure or compressed air or inert gas. The atomisation pressure is such as to form droplet sizes in the region of <20 μm. The droplet sizes of the atomised solution may be determined experimentally for example by a laser diffraction process using a laser made by Sympatec (50 mm focal length, Mie evaluation). Preferably the hot air current from step b) is at temperatures between 100 and 350° C., particularly between 120 and 200° C.

In another preferred embodiment of the process according to the invention, after the drying in step c) spray-dried particles are obtained which have a median aerodynamic diameter of ≦15 μm, preferably ≦10 μm, particularly ≦5 μm. Particularly preferred is a process according to the invention wherein, after the drying in step c), spray-dried particles are obtained which are “inhalable”, i.e. have a diameter which is small enough to allow topical application to the lungs.

The invention further relates to a spray-dried powder formulation containing particles which may be obtained by one of the above mentioned processes according to the invention.

The invention further relates to the use of an organic, physiologically acceptable, sterically demanding acid according to iii) preferably selected from among ascorbic acid, a mono-, di- or trivalent carboxylic acid with the exception of the aminocarboxylic acids, preferably fumaric acid, oxalic acid or succinic acid, and a fruit or culinary acid, preferably citric acid, tartaric acid, malic acid, lactic acid, acetic acid, α-hydroxycaprylic acid or gluconic acid, for stabilising a powder formulation prepared by spray-drying, containing the following components i) to ii):
i) an anticholinergic, preferably a compound of formula 1

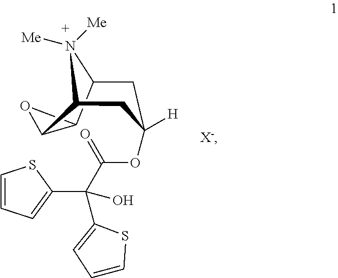

wherein
X⁻ denotes a negatively charged anion.
ii) at least one embedding material selected from among mono- or disaccharides, oligosaccharides, polymers, sugar alcohols and cholesterol.

Particularly preferably the organic, physiologically acceptable, sterically demanding acid according to iii) is used in a concentration in the solution which is to be spray-dried such that the pH obtained for the sprayable solution containing the anticholinergic according to i), preferably a compound of formula 1, and at least one embedding material according to ii) is <7, preferably ≦6, more preferably ≦5, particularly ≦4.

In another particularly preferred embodiment the salt of an organic, physiologically acceptable, sterically demanding acid according to iii), preferably selected from among ascorbate, the salt of a mono-, di- or trivalent carboxylic acid with the exception of the salts of the aminocarboxylic acids, preferably the fumarate, oxalate or succinate, and the salt of a fruit or culinary acid, preferably the citrate, tartrate, malate, lactate, acetate, α-hydroxycapronate or gluconate, is used to stabilise a powder formulation prepared by spray-drying, containing the following components i) to ii):
i) a compound of formula 1

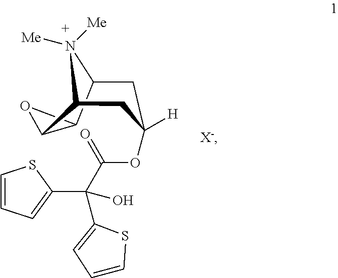

wherein
X⁻ denotes a negatively charged anion.
ii) at least one embedding material selected from among mono- or disaccharides, oligosaccharides, polymers, sugar alcohols and cholesterol.

This additional salt of an organic, physiologically acceptable, sterically demanding acid is preferably used in a concentration such that the molar ratio of the anticholinergic according to i), particularly of the compound according to formula 1, to the cation of the salt is from 1:1 to 1:12, preferably from 1:2 to 1:10 and particularly from 1:3 to 1:8.

In another particularly preferred embodiment a mixture of an organic, physiologically acceptable, sterically demanding acid according to iii) preferably selected from among ascorbic acid, a mono-, di- or trivalent carboxylic acid, with the exception of the aminocarboxylic acids, preferably fumaric acid, oxalic acid or succinic acid, and a fruit or culinary acid, preferably citric acid, tartaric acid, malic acid, lactic acid, acetic acid, α-hydroxycaprylic acid or gluconic acid and the salt of an organic, physiologically acceptable, sterically demanding acid according to iii), preferably selected from among ascorbate, the salt of a mono-, di- or trivalent carboxylic acid with the exception of the salts of the aminocarboxylic acids, preferably the fumarate, oxalate or succinate, and the salt of a fruit or culinary acid, preferably the citrate, tartrate, malate, lactate, acetate, α-hydroxycapronate or gluconate, is used to stabilise a powder formulation prepared by spray-drying, containing the following components i) to ii):
i) a compound of formula 1

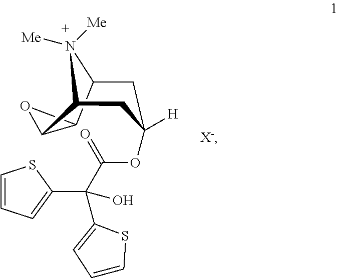

wherein
X⁻ denotes a negatively charged anion, and
ii) at least one embedding material selected from among mono- or disaccharides, oligosaccharides, polymers, sugar alcohols and cholesterol.

It is particularly preferable to use a mixture of citric acid and citrate, preferably an alkali metal citrate, an alkaline earth metal citrate or zinc citrate, particularly sodium citrate, potassium citrate, magnesium citrate, calcium citrate or zinc citrate.

Preferably, the ratio of the organic, physiologically acceptable, sterically demanding acid, preferably citric acid, to the salt of the organic, physiologically acceptable, sterically demanding acid, preferably citrate, in the solution is adjusted such that the solution prepared in step a) has a pH of <7, preferably ≦6, more preferably ≦5, particularly ≦4.

Preferably the concentration of the salt of the organic, physiologically acceptable, sterically demanding acid used is such as to obtain a molar ratio of the anticholinergic according to i), particularly the compound of formula 1, to the cation of the salt of 1:1 to 1:12, preferably from 1:2 to 1:10 and particularly from 1:3 to 1:8.

Preferably X⁻ in the compound of formula 1 denotes an anion selected from among the chloride, bromide, iodide, sulphate, phosphate, methanesulphonate, nitrate, maleate, acetate, citrate, fumarate, tartrate, oxalate, succinate, benzoate and p-toluenesulphonate, particularly bromide.

Preferably, the embedding material ii) used is a mono- or disaccharide selected from among glucose, saccharose, fructose, maltose, lactose, cellobiose and trehalose, an oligosaccharide selected from among oligomaltose, oligofructose, cyclodextrins, dextrins and oligosaccharose, a polymer selected from among inulin, alginate, maltodextrin, starch, starch derivatives, cellulose, cellulose derivatives, PVP (plasdone), gelatine, chitosan, dextranes, pectins, gum arabic, polylactides, poly(lactide-co-glycolides) and polyvinylalcohols, a sugar alcohol selected from among mannitol, xylitol and sorbitolol or cholesterol. However, the particularly preferred embedding materials ii) used are lactose, trehalose and mannitol, particularly lactose.

The present invention also relates to the use of the spray-dried powder formulation according to the invention for preparing a pharmaceutical composition for the treatment of respiratory complaints, particularly for the treatment of COPD and/or asthma, preferably using the inhalers described hereinbefore.

### EXAMPLES

### Formulation Examples

Solutions according to step a) with citric acid:

solution before spray-drying:

| | |
|---|---|
| compound of formula 1 | 0.4 g (1% based on the solid) |
| (X⁻ = Br⁻) | |
| lactose | 40.6 g |
| citric acid | 0.25 g (added to give pH 3; |
| | 0.6% based on the total solid |
| | content of the particles) |
| water | 460 g |

Solutions according to step a) with citric acid and citrate:

solution before spray-drying:

| | |
|---|---|
| compound of formula 1 | 0.4 g (1% based on the solid) |
| (X⁻ = Br⁻) | |
| lactose | 38.5 g |
| citric acid | 2.1 g (added to give pH 3; |
| | 5.2% based on the total solid |
| | content of the particles) |
| zinc citrate dihydrate | 1.0 g (2.5% based on the total solid |
| | content of the particles) |
| | molar ratio of the anticholinergic to |
| | the zinc ion is 1:6 |
| water | 460 g |

### Preparation Examples

Spray-Dried Powder Formulation (With Citric Acid)

### Example 1

### Spray Parameters, Suitable for Producing Inhalable Particles with Ba 679

### Method

The solvent is placed in an Erlenmeyer flask. The embedding material is added batchwise with vigorous stirring (e.g. using a magnetic stirrer) and optionally with heating. As soon as the solution is clear, the pH is adjusted to pH 3 with citric acid and the compound of formula 1 is added. Once it is fully dissolved , spray-drying is carried out immediately. Spray-drying is carried out using a modified BUCHI Mini-Spray Dryer (B-191) in conjunction with a modified 0.5 mm two-substance nozzle and using only N₂ as the process and nozzle gas. Essentially all the glass components have been replaced by metal parts and the aspirator removed. N₂ is fed in as a dry gas (approx. 35 m³/h) through the process gas inlet so that the gas flows through the apparatus in the overpressure range. The outlet filter between the cyclone and aspirator has been removed and the exiting gas directly after the cyclone is diverted into an extractor with an integrated fine particle filter. The two-substance nozzle is made of stainless steel, while the 0.5 mm nozzle cap with mixing needle and nozzle check nut are retained as the central atomising unit. The mass flow of the nozzle gas throughput is determined using an external measuring instrument (Kobold MAS 3015) and uncoupled from the original floating flow meter. Usually, the nozzle is operated at a gas pressure of approx. 6 bar overpressure. The entry temperature of the process gas is 150° C. The mass flow of the spray solution should be selected so as to obtain an outlet temperature of 82±3° C. After the spray drying has ended, the powder has to be removed immediately and stored or further processed in the absence of moisture. The process parameters used are shown in Table 1.

TABLE 1
Spray drying parameters

| volume flow “spraying rate” | 12 | ml/min |
|---|---|---|
| spray pressure (nozzle type) | 6 bar overpressure N₂ (BÜCHI | |
| | spray nozzle 0.5 mm, modified) | |
| volume flow “atomizing pressure” | 2580 litres/h at STP (BÜCHI | |
| (nozzle type) | spray nozzle 0.5 mm, modified) | |
| entry temperature | 150° | C. |
| exit temperature | 80° | C. |
| volume flow “drying gas” | 31 m³/h at STP | |
| cross section of drying tower | 105 | mm |

### Spray-Dried Powder Formulation (With Citric Acid and Citrate)

### Example 2

### Spray Parameters Suitable for Producing Inhalable Particles with Ba 679

### Method

The solvent is placed in an Erlenmeyer flask. The embedding material is added batchwise with vigorous stirring (e.g. using a magnetic stirrer) and optionally with heating. As soon as the solution is clear, the salt of an organic acid is added and the pH is adjusted to pH 3 with citric acid. Then the compound of formula 1 is added. Once it is fully dissolved, spray-drying is carried out immediately. Spray-drying is carried out as described in Example 1. The process parameters used are shown in Table 2.

TABLE 2
Spray-drying parameters

| volume flow “spraying rate” | 12 | ml/min |
|---|---|---|
| spray pressure (nozzle type) | 6 bar overpressure N₂ (BÜCHI | |
| | spray nozzle 0.5 mm, modified) | |
| volume flow “atomizing pressure” | 2580 litres/h at STP (BÜCHI | |
| (nozzle type) | spray nozzle 0.5 mm, modified) | |
| entry temperature | 150° | C. |
| exit temperature | 79° | C. |
| volume flow “drying gas” | 31 m³/h at STP | |
| cross section of drying tower | 105 | mm |

1. A spray-dried powder formulation comprising particles that contain the following components i) to iii):
i) a compound of formula 1 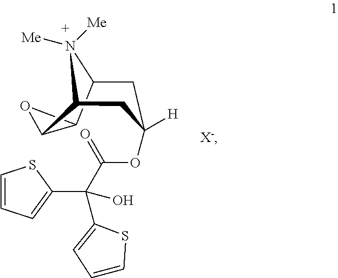
wherein X⁻ is a negatively charged anion;
ii) as an embedding material lactose;
iii) an organic, physiologically acceptable, sterically demanding acid selected from ascorbic acid and a mono-, di- or trivalent carboxylic acid with the exception of the aminocarboxylic acids, wherein the acid is in a concentration such that the sprayable solution has a pH of ≦4.

2. The spray-dried powder formulation according to claim 1, wherein the particles of the spray-dried powder formulation have a mean aerodynamic diameter of less than or equal to 10 μm and are suitable for administration using an inhaler.

3. The spray-dried powder formulation according to claim 1, wherein the compound of formula 1 is in a concentration of between 0.05% and 1%

4. The spray-dried powder formulation according to claim 1, wherein the organic, physiologically acceptable, sterically demanding acid according to iii) is selected from citric acid, tartaric acid, malic acid, lactic acid, acetic acid, α-hydroxycaprylic acid, gluconic acid, fumaric acid, oxalic acid, and succinic acid.

5. The spray-dried powder formulation according to claim 1, wherein the organic, physiologically acceptable, sterically demanding acid according to iii) is citric acid.

6. The spray-dried powder formulation according to claim 1, wherein the spray-dried powder formulation additionally contains the salt of an organic, physiologically acceptable, sterically demanding acid according to iii) selected from ascorbate, the salt of a fruit or culinary acid, and the salt of a mono-, di- or trivalent carboxylic acid, with the exception of the salts of the aminocarboxylic acids.

7. The spray-dried powder formulation according to claim 1, wherein the spray-dried powder formulation additionally contains the salt of an organic, physiologically acceptable, sterically demanding acid according to iii) selected from the salt of a fruit or culinary acid selected from citrate, tartrate, malate, lactate, acetate, α-hydroxycapronate, and gluconate or the salt of a mono-, di- or trivalent carboxylic acid, with the exception of the salts of the aminocarboxylic acids, selected from the fumarate, oxalate, and succinate.

8. The spray-dried powder formulation according to claim 7, wherein the spray-dried powder formulation contains citrate as the salt of the organic, physiologically acceptable, sterically demanding acid according to iii).

9. The spray-dried powder formulation according to claim 7, wherein the salt of the organic, physiologically acceptable, sterically demanding acid is an alkali metal salt, an alkaline earth metal salt or a zinc salt.

10. The spray-dried powder formulation according to claim 1, in which in the compound of formula 1 according to i), X⁻ is an anion selected from the chloride, bromide, iodide, sulphate, phosphate, methanesulphonate, nitrate, maleate, acetate, citrate, fumarate, tartrate, oxalate, succinate, benzoate, and p-toluenesulphonate.

11. The spray-dried powder formulation according to claim 10, in which in the compound of formula 1 according to i), X⁻ is bromide.

12. The spray-dried powder formulation according to claim 1, wherein the embedding material according to ii) is a mono- or disaccharide selected from glucose, saccharose, fructose, maltose, lactose, cellulose, and trehalose.

13. The spray-dried powder formulation according to claim 1, wherein the embedding material according to ii) is an oligosaccharide selected from oligomaltose, oligofructose, cyclodextrins, dextrins, and oligosaccharose.

14. The spray-dried powder formulation according to claim 1, wherein the embedding material according to ii) is a polymer selected from among inulin, alginate, maltodextrin, starch, cellulose, polyvinylpyrrolidone (PVP), gelatine, chitosan, dextranes, pectins, gum arabic, polylactides, poly(lactide-co-glycolides), and polyvinylalcohols.

15. The spray-dried powder formulation according to claim 1, wherein the embedding material according to ii) is a sugar alcohol selected from among mannitol, xylitol, and sorbitol.

16. The spray-dried powder formulation according to claim 1, wherein the organic, physiologically acceptable, sterically demanding acid according to iii) is citric acid and the citric acid is added to the sprayable solution containing the compound according to i), and the at least one embedding material according to ii) in a concentration such that the pH of the solution is ≦4.

17. A powder formulation that contains particles comprising together the following components i) to iii), obtained by a process comprising the following steps:
a) preparing a solution comprising components i) to iii):
i) a compound of formula 1: 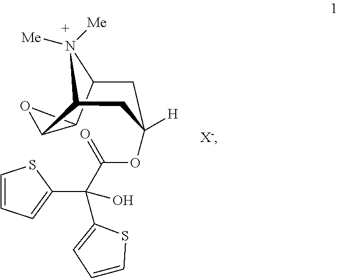
wherein X⁻ is a negatively charged anion, and the compound of formula 1 is in a concentration of between 0.05% and 1%,
ii) at least one embedding material selected from among mono- or disaccharides, oligosaccharides, polymers, sugar alcohols and cholesterol, and
iii) an organic, physiologically acceptable, sterically demanding acid selected from ascorbic acid, a fruit or culinary acid, and a mono-, di-, or trivalent carboxylic acid, with the exception of the aminocarboxylic acids, in a suitable solvent;
b) atomizing the solution from step a) in a hot air current;
c) drying the atomized solution from step b) to form a powder containing particles; and
d) separating off the powder from step c) using a cyclone or filters,
wherein the particles of the spray-dried powder formulation have a mean aerodynamic diameter of less than or equal to 10 μm and are suitable for administration using an inhaler.

18. The powder formulation according to claim 17, wherein in step a) a solution is prepared which contains as the organic, physiologically acceptable, sterically demanding acid according to iii) a fruit or culinary acid selected from citric acid, tartaric acid, malic acid, lactic acid, acetic acid, α-hydroxycaprylic acid, and gluconic acid or a mono-, di-, or trivalent carboxylic acid, with the exception of the aminocarboxylic acids, selected from fumaric acid, oxalic acid, and succinic acid.

19. The powder formulation according to claim 17, wherein in step a) a solution is prepared which contains citric acid as the organic, physiologically acceptable, sterically demanding acid according to iii).

20. The powder formulation according to claim 17, wherein in step a) a solution is prepared which additionally contains the salt of an organic, physiologically acceptable, sterically demanding acid according to iii) selected from ascorbate, the salt of a fruit or culinary acid and the salt of a mono-, di-, or trivalent carboxylic acid, with the exception of the aminocarboxylic acids.

21. The powder formulation according to claim 20, wherein in step a) a solution is prepared which contains as an additional salt of an organic, physiologically acceptable, sterically demanding acid according to iii) the salt of a fruit or culinary acid selected from the citrate, tartrate, malate, lactate, acetate, α-hydroxycapronate, and gluconate or the salt of a mono-, di-, or trivalent carboxylic acid, with the exception of the aminocarboxylic acids, selected from the fumarate, oxalate, and succinate.

22. The powder formulation according to claim 21, wherein in step a) a solution is prepared which additionally contains, as the salt of an organic, physiologically acceptable, sterically demanding acid according to iii), citrate, wherein the citrate is an alkali metal citrate, an alkaline earth metal citrate, or zinc citrate.

23. The powder formulation according to claim 17, wherein in step a) a solution is prepared which comprises a compound of formula 1, wherein X⁻ is an anion selected from the chloride, bromide, iodide, sulphate, phosphate, methanesulphonate, nitrate, maleate, acetate, citrate, fumarate, tartrate, oxalate, succinate, benzoate, and p-toluenesulphonate.

24. The powder formulation according to claim 17, wherein in step a) a solution is prepared which comprises a compound of formula 1 wherein X⁻ is bromide.

25. The powder formulation according to claim 17, wherein the embedding material ii) of the solution from step a) is a mono- or disaccharide selected from glucose, saccharose, fructose, maltose, lactose, cellobiose and trehalose or an oligosaccharide selected from oligomaltose, oligofructose, cyclodextrins, dextrins, and oligosaccharose, or a polymer selected from inulin, alginate, maltodextrin, starch, cellulose, PVP, gelatine, chitosan, dextranes, pectins, gum arabic, polylactides, poly(lactide-co-glycolides), and polyvinylalcohols, or a sugar alcohol selected from mannitol, xylitol, and sorbitolol or cholesterol.

26. The powder formulation according to claim 17, wherein the atomizing according to step b) is carried out using a nozzle.

27. The powder formulation according to claim 17, wherein the hot air current according to step b) is at temperatures between 100° C. and 350° C.

28. The powder formulation according to claim 17, wherein in step a) a solution is prepared with sufficient acid, component iii), so that the solution has a pH of ≦5.

29. The powder formulation according to claim 17, wherein the hot air current according to step b) is at temperatures between 120° C. and 200° C.

30. The powder formulation according to claim 1, wherein the spray-dried powder is packed as an individual dose into a capsule.

31. The powder formulation according to claim 17, wherein the spray-dried powder is packed as an individual dose into a capsule.
